(19) United States
(12) Patent Application Publication
(10) Pub. No.: US 2021/0145927 A1
(43) Pub. Date: May 20, 2021

(54) OIL-FREE AND FAT-FREE AQUEOUS SUSPENSIONS OF CYCLOSPORIN

(71) Applicant: NEWPORT RESEARCH, INC., Newport Beach, CA (US)

(72) Inventor: Harun Takruri, Newport Beach, CA (US)

(21) Appl. No.: 17/160,270

(22) Filed: Jan. 27, 2021

Related U.S. Application Data
(63) Continuation of application No. 14/597,948, filed on Jan. 15, 2015, now abandoned.
(60) Provisional application No. 62/009,055, filed on Jun. 6, 2014.

(51) Int. Cl. A61K 38/13; A61K 47/32; A61K 9/00; A61K 9/10

(52) U.S. Cl. CPC A61K 38/13; A61K 9/10; A61K 9/0048; A61K 47/32

(57) ABSTRACT

Compositions that are oil-free and fat-free aqueous suspensions of cyclosporin and contain a cyclosporin (e.g., cyclosporine), a hydrophilic pharmaceutically acceptable solvent in which the cyclosporin (e.g., cyclosporine) is soluble, a dispersing agent, a suspending agent and an aqueous vehicle are disclosed. Methods of producing such compositions, as well as methods of using the compositions to treat ophthalmic disorders are also disclosed.

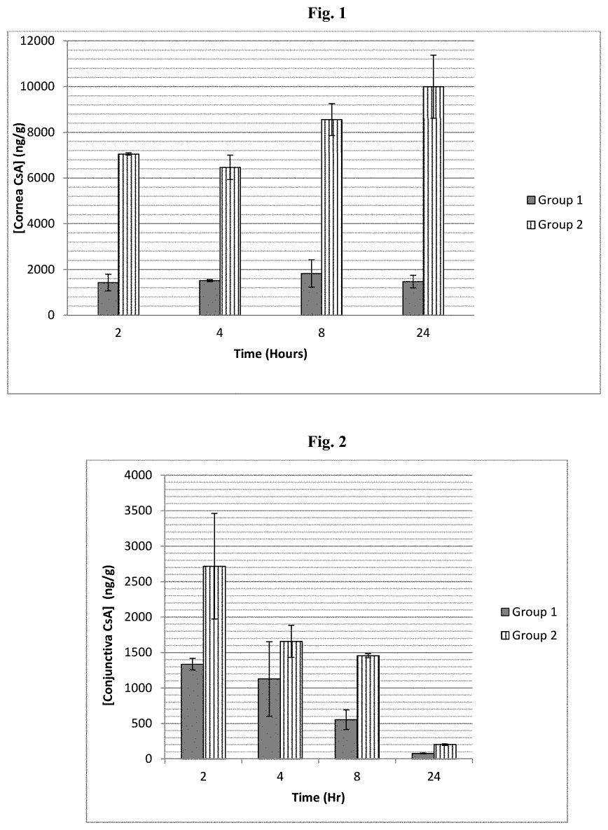

### CROSS-REFERENCE

This application claims the benefit priority of U.S. Provisional Application No. 62/009,055, filed Jun. 6, 2014, which application is incorporated herein by reference in its entirety.

### FIELD OF THE INVENTION

The invention relates generally to formulations of cyclosporin (e.g., cyclosporine), and more specifically to oil-free and fat-free, aqueous suspensions of cyclosprorin.

### BACKGROUND OF THE INVENTION

The low solubility of cyclosporins in water (e.g., below 0.004% for cyclosporine) makes it difficult to develop therapeutically active solutions of this drug, particularly for ophthalmic use. Thus, alternate formulations have been developed for systemic and topical use based on its solubility in oils and surfactants. For example, formulations incorporating cyclosporine have been prepared as oily solutions containing ethanol. However, if oily preparations containing cyclosporine are applied directly to the eyes, irritation or a clouding of visual field may result. A further drawback of formulations containing a high concentration of oils is that oils can exacerbate the symptoms of certain ocular surface diseases such as dry eye, which is treated with cyclosporine. Therefore, these oily formulations may not be clinically acceptable. Additionally, these formulations often suffer from physical instability due to cyclosporin's propensity to undergo conformational change and crystallize out. The crystallization problem has been noticed in formulations containing corn oil or medium chain triglycerides. More recent formulations are emulsions, where cyclosporine is dissolved in oil, which then is emulsified in water with the aid of surfactants and polymers.

### SUMMARY OF THE INVENTION

Because cyclosporine is soluble in oil, current formulations of cyclosporine for topical delivery to the eye use oil as a solvent. Given that the residence time of a formulation topically applied to the eye is short, due to the cyclosporine being washed out, such a formulation poses a problem for delivery of cyclosporine to the eye. In particular, the cyclosporine must diffuse out of the oily solvent, in which it is soluble, and into the hydrophilic environment of the cornea, where it is less soluble, in order to contact the cornea prior to the formulation being washed out. The present compositions address this problem by providing amorphous particles of cyclosporin (e.g., cyclosporine) in a hydrophilic solvent, which will increase contact of the cyclosporin with the cornea. Accordingly, provided herein are pharmaceutical compositions in the form of suspensions of cyclosporin (e.g., cyclosporine) suitable for use in the eye, ear, and nose, and particularly in the eye. The present compositions are not emulsions and contain no oils or fats. The compositions contain pharmaceutically acceptable solvents for cyclosporins, which do not require removal from the final product, a dispersing agent, and a suspending agent. The compositions are compatible with antimicrobial preservatives such as benzalkonium chloride.

Accordingly, in one aspect, there are provided compositions including a cyclosporin; a hydrophilic pharmaceutically acceptable solvent in which cyclosporin is soluble; a dispersing agent; a suspending agent; and an aqueous vehicle, wherein the solvent, dispersing agent, suspending agent, and vehicle are each oil-free and fat-free, and wherein the composition is a suspension of cyclosporin and is oil-free and fat-free. In some embodiments, the cyclosporin is cyclosporine. In some embodiments, the cyclosporine particles are amorphous particles.

In another aspect, there are provided methods of treating an ophthalmic disorder by contacting an affected eye of a patient having the ophthalmic disorder with the cyclosporin (e.g., cyclosporine) compositions described herein, wherein the disorder is selected from the group consisting of dry eye syndrome, anterior or posterior uveitis, chronic keratitis, keratoconjunctivitis sicca, vernal keratoconjunctivitis, phacoanaphylactic endophthalmitis, atopic keratoconjunctivitis, conjunctivitis, vernal conjunctivitis, keratoplasty, immunoreactive graft rejection post corneal transplantation, Behcet disease, Mooren's ulcer, ocular pemphigus, and rheumatoid ulcer.

In yet another aspect, there are provided methods of producing an oil-free, fat-free cyclosporin suspension by mixing (a) a solution of a cyclosporin dissolved in a hydrophilic pharmaceutically acceptable solvent in which cyclosporin is soluble, and (b) a composition comprising a dispersing agent, a suspending agent, and an aqueous vehicle, wherein the solution and the composition are each oil-free and fat-free, thereby producing a suspension that is oil-free and fat-free and having cyclosporin particles of 20 μm or less dispersed in the aqueous vehicle. In some embodiments, the cyclosporin is cyclosporine.

### DETAILED DESCRIPTION OF THE INVENTION

Current formulations of cyclosporine for topical delivery to the eye use oil as a solvent because cyclosporine is soluble in oil, whereas it is less soluble in water. Given that the residence time of a formulation topically applied to the eye is short, due to the cyclosporine being washed out, such a formulation poses a problem for delivery of cyclosporine to the eye. In particular, the cyclosporine must diffuse out of the oily solvent, in which it is soluble, and into the hydrophilic environment of the cornea, where it is less soluble, in order to contact the cornea prior to the formulation being washed out. The present compositions address this problem by providing amorphous particles of cyclosporin (e.g., cyclosporine) in a hydrophilic solvent, which will increase contact of the cyclosporin with the cornea. The present invention utilizes the high solubility of cyclosporin (e.g., cyclosporine) in organic, pharmaceutically acceptable, hydrophilic solvents to produce concentrated solutions of cyclosporin, which are then added to aqueous vehicles resulting in stable colloidal dispersions of cyclosporin suitable for use in the eye, ear, or nose. Whereas the solubility of cyclosporine in water is about 30 μg/mL, it is about 200 mg/mL in ethanol, 400 mg/g in propylene glycol, and 260 mg/g in polyethylene glycol 400. The invention formulation does not require removal of the organic solvent from the colloidal dispersion, and rather it becomes an integral part of the formulation contributing to osmolarity and in some instances, antimicrobial properties. Thus, in some embodiments, therapeutically useful levels of 0.005 to 1.0% cyclosporine are easily prepared while maintaining the level of the organic solvent in the acceptable range of tolerability.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

The cyclosporins comprise a large and recognized class of peptide compounds having pharmaceutical utility, for example, immunosuppressant, anti-inflammatory, and/or anti-parasitic activity and/or activity in abrogating tumor resistance to anti-neoplastic or cytostatic drug therapy. The cyclosporins include, for example, naturally occurring fungal metabolites, such as cyclosporin A, B, C, D and G, as well as a wide variety of synthetic and semi-synthetic cyclosporins, for example, the dihydro- and iso-cyclosporins (see e.g. U.S. Pat. Nos. 4,108,985; 4,210,581 and 4,220,641), [(D)-Ser]⁸-Ciclosporin (see U.S. Pat. No. 4,384,996), [0-acetyl, (D)-Ser]⁸-Ciclosporin (see U.S. Pat. No. 4,764,503), [β-fluoro-(D)Ala]⁸-Ciclosporin (see UK Patent Application 2,206,119A), [Val]²-[(D)methylthio-Sar]³- and [Dihydro-MeBmt]¹-[Val]²-[(D)methylthio-Sar]³-Ciclosporin [see U.S. Pat. No. 4,703,033], [0-(2-hydroxyethyl)-(D)Ser]⁸-Ciclosporin, and [3′-deshydroxy-3′-keto-MeBmt]¹-[Val]²-Ciclosporin and many more. The most widely investigated cyclosporin is cyclosporin A. The terms “cyclosporin A,” “cyclosporine A” and “cyclosporine” are used interchangeably herein. Cyclosporin A has been shown to suppress selectively a variety of T-lymphocyte functions, including prevention of maturation and expression of sensitized T-lymphocytes in cell mediated immune responses, and is now successfully and widely used in the suppression of organ transplant rejection. Cyclosporin A has also been used systemically in the treatment of intraocular inflammatory or autoimmune diseases, such as uveitis. Accordingly, in some embodiments, the cyclosporin used in the invention compositions is a naturally ocurring cyclosporin, a synthetic cyclosporin, or a semi-synthetic cyclosporin. In particular embodiments, the cyclosporin is cyclosporine.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20% or ±10%, or ±5%, or even ±1% from the specified value, as such variations are appropriate for the disclosed compositions or to perform the disclosed methods.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In one aspect, there are provided compositions including a cyclosporin; a hydrophilic pharmaceutically acceptable solvent in which the cyclosporin is soluble; a dispersing agent; a suspending agent; and an aqueous vehicle, wherein the solvent, dispersing agent, suspending agent, and vehicle are each oil-free and fat-free, and wherein the composition is a suspension of cyclosporin and is oil-free and fat-free. In particular embodiments, the cyclosporin is cyclosporine and the hydrophilic pharmaceutically acceptable solvent is one in which cyclocylosporine is soluble.

As used herein, the terms “oil” and “fat” refer to a substance or mixture of substances that is used or can be used as a pharmaceutical excipient, and that is very slightly soluble or insoluble in water (as defined in the USP), and has no significant surface activity in aqueous systems. Oils and fats can be mineral, synthetic, animal, or plant in origin. Exemplary oils and fats include: hydrocarbons, such as mineral oil and paraffin; alcohols, such as cetyl alcohol; acids, such as oleic and stearic acid and other saturated and unsaturated fatty acids; synthetic esters such as ethyl oleate and isopropyl myristate; triglyceride esters such as olive, peanut, sesame, castor oils, and short and medium chain mono-, di-, and triglycerides; waxes, such as beeswax and spermaceti; essential oils, such as rose, fennel, anise, peppermint and lemon oils; organic silicones, such as dimethicone and simethicone; and any other substance or mixture of substances meeting the criteria above.

The above definition of oils and fats does not include phospholipids, such as lecithin, and water-insoluble, surface active agents, such as sorbitan monooleate because they do not satisfy the above definition, and which, in some embodiments, are used as dispersing agents, by virtue of their surface activity. Similarly, components such as glycerin, PEG, and polyoxyl 15 hydroxystearate are water- soluble, and carbomer homo- and copolymers disperse/hydrate readily in water, and thus, are not oils or fats as defined herein.

In some embodiments, the oil-free and fat-free compositions contain about 0.1% oils and/or fats. In some embodiments, oil-free and fat-free compositions contain less than about 0.1% oils and/or fats. In some embodiments, oil-free and fat-free compositions contain less than about 0.05% oils and/or fats. In some embodiments, oil-free and fat-free compositions contain less than about 0.025% oils and/or fats. In some embodiments, oil-free and fat-free compositions contain less than about 0.01% oils and/or fats.

The term “dispersion” as used herein refers to a dispersed system having at least two phases: the substance that is dispersed, known as the dispersed phase (or internal phase), and the phase in which that substance is dispersed, known as the continuous phase (or external phase or dispersion medium). Suspensions and emulsions are examples of dispersions. Based on the particle size of the dispersed phase, dispersions are generally classified as molecular dispersions (i.e., solutions), colloidal dispersions, or coarse dispersions. It is commonly accepted that molecular dispersions have dispersed particles lower than 1 nm in size; colloidal dispersions have particle sizes between 1 nm and 1 μm in size; and coarse dispersions have particles greater than 1 μm in size.

The term “suspension” as used herein refers to a dispersed system in which a finely divided solid (i.e., the dispersed phase) is dispersed uniformly in a liquid dispersion medium (i.e., the continuous phase). Suspensions are further classified as course or colloidal depending on the particle size of the dispersed phase. For example, suspensions with a particle size greater than about 1 μm are classified as coarse suspensions, while those having particles that are less than 1 μm are classified as colloidal suspensions, also called “nanosuspensions.” It is desired that the internal phase be dispersed uniformly in the dispersion medium and not sediment or settle during storage, however this is difficult to achieve due to the thermodynamic instability of a suspension. Settling of a suspension requires resuspension of the dispersed phase by, for example, shaking or agitation of the composition prior to application. The prevention of settling is important for preservative-free unit dose products, where shaking is not feasible due to the small size/volume. Therefore, suspending agents that increase the viscosity and prevent the particles from settling out are used in some embodiments of the disclosed compositions. Additional stability is obtained by the use of dispersing agents that prevent the primary particles from aggregation to form larger particles susceptible to settling.

The term “emulsion” as used herein refers to a dispersed system in which a finely divided liquid (i.e., the dispersed phase) is dispersed uniformly in another liquid dispersion medium (i.e., the continuous phase) using an emulsifier. For example, in an oil-in-water emulsion, the dispersed phase is an oil, and the dispersion medium is aqueous.

In particular embodiments, the compositions are ophthalmic compositions, that is, the compositions are suitable for ophthalmic use. The ophthalmic compositions are formulated as eye-drop formulations in some embodiments. In some embodiments, the ophthalmic compositions are filled in appropriate containers to facilitate administration of the composition to the eye, for example, a plastic bottle with control dropper tip. Accordingly, in another aspect there are provided ophthalmic compositions as defined above in a container appropriate for ophthalmic application of the composition, for example, appropriate for application of the ophthalmic composition to or at the surface of the eye (e.g., to the cornea or conjunctiva). In some embodiments, the ophthalmic composition is an aqueous gel. In some embodiments, such aqueous gels are formulated by increasing the concentration of suspending agent (e.g., carbomer homopolymer or carbomer copolymer) to achieve a semi-solid consistency. In some embodiments, the ophthalmic aqueous gel composition is filled into an ophthalmic ointment tube.

In some embodiments of the compositions, the cyclosporin is in a therapeutically effective amount. As used herein, a “therapeutically effective amount” or an “effective amount” is an amount of a cyclosporin or a composition thereof sufficient to effect beneficial or desired clinical results including reduction or amelioration of symptoms stemming from the disorder or condition being treated. The skilled artisan can readily determine a therapeutically effective amount of a given cyclosporin for a particular indication. In some embodiments, the cyclosporin is cyclosporine and is in the composition in an amount between about 0.005 to 1.0% w/v. In particular embodiments, the cyclosporine is in an amount of about 0.005 to about 0.1% (w/v), or from about 0.05 to about 0.1% (w/v), or from about 0.01 to about 0.075% (w/v). In some embodiments, the cyclosporine is in the composition at concentration of about 0.05% (w/v). As used herein, 1% (w/v) is equivalent to 1 g per 100 mL.

The compositions contain the pharmaceutically acceptable solvent in which the cyclosporin (e.g., cyclosporine) is soluble. The term “pharmaceutically acceptable,” when used in reference to a solvent, vehicle, or excipient, means that the solvent, vehicle, or excipient must be compatible with the other ingredients of the formulation and not deleterious to the recipient thereof. Such a component is one that is suitable for use with humans or animals without undue adverse side effects. Non-limiting examples of adverse side effects include toxicity, irritation, and/or allergic response. Such pharmaceutically acceptable solvents are organic. Further, the cyclosporin (e.g., cyclosporine) is highly soluble in these solvents in order to produce a concentrated solution of cyclosporin. This high solubility allows the formulation of a composition having an effective amount of cyclosporin, as well as allowing the solvent to remain in the final composition because it will be in a low enough amount that it causes little to no irritation upon application of the final composition to a patient's eye. Appropriate solvents for use in the present compositions have a solubility of at least 200 mg cyclosporine per mL of solvent, or at least 100 mg/mL, or at least 50 mg/mL, or even at least 10 mg/mL.

In some embodiments, the cyclosporin is cyclosporine and the hydrophilic pharmaceutically acceptable solvent is one in which cyclocylosporine is soluble. In some embodiments, the pharmaceutically acceptable solvent in which the cyclosporin (e.g., cyclosporine) is soluble is selected from the group consisting of ethanol, propylene glycol, polyethylene glycol, glycerin, benzyl alcohol, polysorbates, tyloxapol, poloxamers, acetone, DMSO, hydrophilic surfactants that are solid at room temperature and act as a solvent when melted at a higher temperature, and combinations thereof. In some embodiments, the pharmaceutically acceptable solvent in which the cyclosporin (e.g., cyclosporine) is soluble is selected from the group consisting of ethanol, propylene glycol, polyethylene glycol, glycerin, benzyl alcohol, polysorbates, tyloxapol, poloxamers, hydrophilic surfactants that are solid at room temperature and act as a solvent when melted at a higher temperature, and combinations thereof. In particular embodiments, the solvent is polyethylene glycol. In other embodiments, the solvent is alcohol, particularly ethanol. In some embodiments, the hydrophilic surfactant that is solid at room temperature is polyoxyl 15 hydroxystearate.

In some embodiments, the concentration of pharmaceutically acceptable solvent in the compositions is within the range of from about 0.05% to about 10.0% (w/v), or from about 0.1 to about 5.0% (w/v), or from about 0.1% to about 2.5% (w/v).

In some embodiments, a dispersing agent is used in the formulation to disaggregate the precipitated particles upon contact with the aqueous vehicle (e.g., “Part 2” of Example 1). In some embodiments, the dispersing agent is a surfactant. In some embodiments, the surfactant is selected from the group of surface active agents that are primarily nonionic and include without limitation polysorbate 80, polysorbate 60, polysorbate 40, polysorbate 20, polyoxyl 40 stearate, polyoxyl 15 hydroxystearate, poloxamers, tyloxapol, POE 35 castor oil, and combinations thereof. It is to be appreciated that any similar pharmaceutically acceptable surface active agents is usable at levels that do not cause irritation or discomfort when applied to the eye, ear, or nose. Accordingly, in some embodiments, the surfactant is selected from the group consisting of polysorbate 80, polysorbate 60, polysorbate 40, polysorbate 20, polyoxyl 40 stearate, polyoxyl 15 hydroxystearate, poloxamers, tyloxapol, POE 35 castor oil, and other pharmaceutically acceptable hydrophilic surfactants. In particular embodiments, the pharmaceutically acceptable hydrophilic surfactant is an anionic surfactant or a cationic surfactant. In some embodiments, the anionic surfactant is sodium lauryl sulfate or docusate sodium. In other embodiments, the cationic surfactant is benzalkonium chloride.

In some embodiments, the concentration of dispersing agent in the compositions is within the range of from about 0.005% to about 5.0% (w/v), or from about 0.01 to about 2.0% (w/v), or from about 0.01% to about 0.5% (w/v).

In some embodiments, a suspending agent is used to increase the viscosity and enhance the physical stability of the colloidal dispersion. In some embodiments, suspending agents are polymers that are synthetic, semi-synthetic, or natural, and include without limitation: carbomer homopolymers, carbomer copolymers, carbomer interpolymers, polycarbophil, soluble cellulose derivatives such as carboxymethylcellulose sodium (NaCMC), hydroxyethylcellulose, hypromellose and others; polyvinyl alcohol, povidone, hyaluronic acid and its salts, chondroitin sulfate, gellan and other natural gums, and other pharmaceutically acceptable polymers. Suspending agents might also provide some surfactant properties as noted above. Accordingly, in some embodiments, the suspending agent in the composition is selected from the group consisting of carbomers, soluble cellulose derivatives, polyvinyl alcohol, povidone, hyaluronic acid and its salts, chondroitin sulfate, gellan, and other natural gums. In some embodiments, the soluble cellulose derivative is selected from the group consisting of carboxymethylcellulose sodium (NaCMC), hydroxyethylcellulose, and hypromellose. In particular embodiments, the suspending agent is a carbomer homopolymer. In some embodiments, the suspending agent is a carbomer copolymer.

In some embodiments, the concentration of suspending agent in the compositions is within the range of from about 0.005% to about 5.0% (w/v), or from about 0.01 to about 2.0% (w/v), or from about 0.01% to about 0.5% (w/v). In some embodiments, the suspending agent is at a concentration sufficient to achieve a semi-solid consistency (e.g., to form an aqueous gel).

In some embodiments, the aqueous vehicle is any pharmaceutically acceptable aqueous vehicle commonly used in ophthalmic formulations. In some embodiments, the aqueous vehicle is selected from the group consisting of water, saline, and phosphate buffered saline. In particular embodiments, the aqueous vehicle is water.

The compositions may further include one or more excipients. Such excipients are pharmaceutically acceptable components. In some embodiments, the one or more excipients are selected from the group consisting of glycerin, mannitol, sodium chloride, tonicity adjusters, buffers, pH adjusters, chelating agents, and antioxidants. pH adjusters include pharmaceutically acceptable acids or bases. In some embodiments, the pH adjuster is sodium hydroxide. In other embodiments, the pH adjuster is hydrochloric acid. In some embodiments, the tonicity adjuster is mannitol. In some embodiments the chelating agent is edetate disodium.

The compositions may further contain an effective amount of an antimicrobial preservative. In some embodiments, any suitable preservative or combination of preservatives is employed. The amounts of preservative components included in the present compositions are sufficient to be effective in preserving the compositions and can vary based on the specific preservative component employed, the specific composition involved, the specific application involved, and the like factors. In some embodiments, preservative concentrations are in the range of about 0.00001% to about 0.5% (w/v) of the composition. In some embodiments, other concentrations of certain preservatives are employed, as the skilled artisan can readily ascertain an effective amount of preservative for a given formulation.

Examples of suitable preservatives include, without limitation, benzalkonium chloride, methyl and ethyl parabens, hexetidine, phenyl mercuric salts and the like and mixtures thereof. Thus, in some embodiments, the preservatives include quaternary ammonium salts such as benzalkonium chloride and cetrimide, chlorobutanol, sorbic acid, boric acid, methyl and ethyl parabens, hexetidine, phenyl mercuric salts and any other preservatives known to be safe and effective when used in topical products, and mixtures thereof. In particular embodiments, the preservative is benzalkonium chloride.

Other useful preservatives include antimicrobial peptides. In some embodiments, the antimicrobial peptides include, without limitation, defensins, peptides related to defensins, cecropins, peptides related to cecropins, magainins and peptides related to magainins and other amino acid polymers with antibacterial, antifungal and/or antiviral activities. Mixtures of antimicrobial peptides or mixtures of antimicrobial peptides with other preservatives are also included within the scope of the present invention.

The compositions and methods are independent of pH. Any pH can be selected that does not impact the chemical stability of cyclosporine and is tolerated by the patient upon application. An appropriate pH is readily ascertained by the skilled artisan. In some embodiments, the pH is from about 4.0 to about 9.0. In other embodiments, the pH is from about 5.0 to about 8.0, or from about 6.0 to about 8.0.

The present compositions are suspensions of cyclosporin (e.g., cyclosporine), that is, cyclosporin is the dispersed phase. In some embodiments, the cyclosporin is dispersed in particles of 20 μm or less. In other embodiments, the cyclosporin is dispersed in particles of 5 μm or less. In still other embodiments, the cyclosporin is dispersed in particles of 1 μm or less. In still other embodiments, the cyclosporin is dispersed in particles of 1 nm to 1 μm. In yet other embodiments, the cyclosporin is dispersed in particles of 10 nm to 500 nm. In further embodiments, the cyclosporin is dispersed in particles of 50 nm to 300 nm. In any of the preceding embodiments, the cyclosporin is in amorphous particles. In some embodiments, the particles are non-aggregating. In any of the preceding embodiments, the cyclosporin is cyclosporine.

The ophthalmic compositions are useful for the same indications as other topical ophthalmic compositions containing cyclosporin (e.g., cyclosporine), for example diseases affecting the cornea, the aqueous, the lens, the iris, the ciliary, the choroid or the retina. The ophthalmic compositions are useful particularly for the treatment of an autoimmune or inflammatory disease or condition of the eye or of the surrounding or associated organs or tissues, which has undesirably elevated immune response or inflammatory reaction or event as part of its etiology. In some embodiments, the ophthalmic compositions are used for treating the anterior or posterior segment of the eye. For example, in some embodiments, the compositions are used for the treatment of dry eye syndrome, anterior or posterior uveitis, chronic keratitis, keratoconjunctivitis sicca, vernal keratoconjunctivitis, phacoanaphylactic endophthalmitis, atopic keratoconjunctivitis, conjunctivitis, including vernal conjunctivitis, or in keratoplasty. The ophthalmic compositions may also be used in the treatment of immunoreactive graft rejection post corneal transplantation, Behcet disease, and autoimmune corneal diseases such as Mooren's ulcer, ocular pemphigus, and rheumatoid ulcer.

Accordingly, there are provided methods of treating an ophthalmic disorder in a patient by contacting an affected eye of a patient having the ophthalmic disorder with the disclosed cyclosporin compositions, wherein the disorder is selected from the group consisting of dry eye syndrome, anterior or posterior uveitis, chronic keratitis, keratoconjunctivitis sicca, vernal keratoconjunctivitis, phacoanaphylactic endophthalmitis, atopic keratoconjunctivitis, conjunctivitis, vernal conjunctivitis, keratoplasty, immunoreactive graft rejection post corneal transplantation, Behcet disease, Mooren's ulcer, ocular pemphigus, and rheumatoid ulcer. In some embodiments, the disorder is selected from the group consisting of dry eye syndrome, phacoanaphylactic endophthalmitis, uveitis, vernal conjunctivitis, atopic keratoconjunctivitis, and corneal graft rejection, thereby treating the disorder. In some embodiments, the disorder is dry eye. In some embodiments, the cyclosporin is cyclosporine.

As used herein, “treatment” or “treating” is an approach for obtaining beneficial or desired clinical results. For purposes of this invention, beneficial or desired clinical results include, but are not limited to, reduction or amelioration of symptoms stemming from the disorder or condition being treated.

In yet another aspect, there are provided methods of producing an oil-free, fat-free cyclosporin suspension by mixing (a) a solution of a cyclosporin dissolved in a hydrophilic pharmaceutically acceptable solvent in which the cyclosporin is soluble, and (b) a composition comprising a dispersing agent, a suspending agent, and an aqueous vehicle, wherein the solution and the composition are each oil-free and fat-free, thereby producing a suspension that is oil-free and fat-free and having cyclosporin particles of 20 μm or less dispersed in the aqueous vehicle. In particular embodiments, the cyclosporin is cyclosporine and the hydrophilic pharmaceutically acceptable solvent is one in which cyclocylosporine is soluble.

In general, the suspensions provided herein are prepared by adding the dispersed phase (e.g., a solution of cyclosporin dissolved in a relatively small volume of the hydrophilic solvent) to a large volume of the dispersion medium (e.g., aqueous vehicle pre-mixed with suspending agent and dispersing agent, and any other desired agents or components). In some embodiments, the dispersed phase of the suspension is prepared by dissolving the cyclosporin (e.g., cyclosporine) in a sufficient volume of the hydrophilic solvent to solubilize the cyclosporin. For hydrophilic solvents that are solid at room temperature, these solvents are heated to a temperature sufficient to melt the solid, and then the cyclosporin is dissolved in the liquid form of the solvent. In some embodiments, a suitable temperature for preparation of a composition is determined by routine experimentation. Where the hydrophilic solvent is a liquid at room temperature, no heating is necessary. The dispersion medium is prepared by dissolving the suspending agent, the dispersing agent, and any other optional components such as preservatives or excipients into an appropriate volume of aqueous vehicle.

Methods of mixing the phases are well-known in the art and can employ a mixer such as an OMNI stator-rotor mixer or equivalent. In some embodiments, the size of the cyclosporin particles produced depends on the batch processing temperature.

In some embodiments, the compositions of the present invention are sterilized by preparing two sterile parts and combining them aseptically. For example, in some embodiments, the first part (Part 1) is the solution of cyclosporine in the designated solvent(s) and is sterilized by filtration using, for example, a 0.22 micron filter; and the second part (Part 2) consists of the remaining components and is sterilized using heat (e.g., autoclave steam sterilization) or, if the viscosity is low enough, sterile filtration using 0.22 micron filters. This procedure minimizes exposure of cyclosporine to potential degradation by heat. However, in certain embodiments the complete formulation is sterilizable by autoclaving without undue effect on the stability of cyclosporine.

The following examples are intended to illustrate but not limit the invention. In these examples “Carbomer Homopolymer Type B” refers to CARBOPOL 974P NF carbomer homopolymer type B (manufactured by Lubrizol).

### EXAMPLE 1

| Ingredient | % |
|---|---|
| Cyclosporine | 0.10 |
| Polyethylene Glycol 300 | 2.0 |
| Carbomer Homopolymer Type B | 0.20 |
| Tyloxapol | 0.025 |
| Glycerin | 2.0 |
| Sodium Chloride | 0.03 |
| Sodium Hydroxide | qs, pH 7.2-7.4 |
| Purified Water | qs. 100 |

A batch of the above formulation was prepared by the following method:

Part 1 consisted of cyclosporine dissolved in PEG 300 at ambient room temperature.

Part 2 consisted of the remaining ingredients prepared by dispersing carbomer in water, followed by the addition of the rest of the ingredients and pH adjustment with sodium hydroxide to the desired pH.

A stator-rotor OMNI mixer was introduced in Part 2, and, while mixing; Part 1 was added slowly to completion. A stable colloidal dispersion was obtained and submitted for particle size analysis using Horiba LA950 laser light scattering instrument. The mean particle size obtained was 180 nanometers. After 13.5 months storage at 2-8° C. of an unautoclaved sample, the mean particle size was 1.35 microns.

A sample of the batch was autoclaved at 121° C. for 30 minutes. The mean particle size for this sample was 2.322 microns. This suggested that the particle size was a function of the processing temperature selected. After 13.5 months storage at ambient room temperature the mean particle size of this sample was 2.377 microns. The stability of particle size in the autoclaved sample was remarkable, and may have been due to an annealing and stabilizing effect of temperature. In contrast, the increase in particle size in the unautoclaved sample may have been due to the higher solubility of cyclosporine at lower temperatures leading to some Ostwald ripening.

### EXAMPLE 2

| Ingredient | % |
|---|---|
| Cyclosporine | 0.10 |
| Polyethylene Glycol 300 | 2.0 |
| Carbomer Homopolymer Type B | 0.06 |
| Tyloxapol | 0.025 |
| Glycerin | 2.0 |
| Sodium Hydroxide | qs, pH 7.2-7.4 |
| Purified Water | qs. 100 |

The batch was prepared as in Example 1. The mean particle size measured was 138 nanometers.

### EXAMPLE 3

In this example, a high concentration (0.5%) of cyclosporine was used. The formulation did not require glycerin as tonicity adjuster because PEG 300 at 8% served as solvent for cyclosporine and tonicity adjuster for the final formulation.

| Ingredient | % |
|---|---|
| Cyclosporine | 0.50 |
| Polyethylene Glycol 300 | 8.0 |
| Carbomer Homopolymer Type B | 0.07 |
| Polysorbate 80 | 0.10 |
| Sodium Hydroxide | qs, pH 7.2-7.4 |
| Purified Water | qs. 100 |

The batch was prepared as in Example 1. The mean particle size measured was 183 nanometers.

### EXAMPLE 4

In this example, the formulation was designed to show that while it may be preferred to have a surfactant in the formulation to prevent aggregation of the primary particles, a colloidal dispersion could be obtained without it.

| Ingredient | % |
|---|---|
| Cyclosporine | 0.10 |
| Polyethylene Glycol 300 | 2.0 |
| Carbomer Homopolymer Type B | 0.07 |
| Mannitol | 4.0 |
| Sodium Hydroxide | qs, pH 7.2-7.4 |
| Purified Water | qs. 100 |

The batch was prepared as in Example 1. The mean particle size measured as function of sonication time in the instrument was as follows:

| Sonication Time | Particle Size |
|---|---|
| 90 seconds | 409 nanometers |
| 150 seconds | 279 nanometers |
| 210 seconds | 234 nanometers |

### EXAMPLE 5

In this example, a low concentration of alcohol, USP was as a solvent for cyclosporine.

| Ingredient | % |
|---|---|
| Cyclosporine | 0.05 |
| Alcohol | 1.0 |
| Carbomer Homopolymer Type B | 0.07 |
| Tyloxapol | 0.025 |
| Glycerin | 0.72 |
| Sodium Hydroxide | qs, pH 7.2-7.4 |
| Purified Water | qs. 100 |

The batch was prepared as in Example 1. The mean particle size measured was 2.19 microns.

### EXAMPLE 6

In this example, a carbomer copolymer (PEMULEN TR-2 polymer, manufactured by Lubrizol) is used instead of Carbomer Homopolymer Type B used in Example 1.

| Ingredient | % w/v |
|---|---|
| Cyclosporine | 0.10 |
| Polyethylene Glycol 300 | 2.0 |
| Carbomer Copolymer Type A | 0.10 |
| Polysorbate 80 | 0.10 |
| Glycerin | 2.0 |
| Sodium Hydroxide | qs, pH 7.0-7.4 |
| Purified Water | qs. 100.00 |

Part 1 consisted of cyclosporine dissolved in PEG 300 at ambient room temperature.

Part 2 consisted of the remaining ingredients prepared by dispersing carbomer coplymer in water, followed by the addition of the rest of the ingredients and pH adjustment with sodium hydroxide to the desired pH.

The batch was prepared as in Example 1. The mean particle size measured was 463 nanometers. The mean particle size after 8 months of storage at ambient room temperature was 1.144 microns.

### EXAMPLE 7

In this example, polyoxyl 15 hydroxystearate (SOLUTOL HS 15 polyoxyl 15 hydroxystearate, manufactured by BASF) is used both as a solvent at high temperature and as the surfactant/dispersing agent.

| Ingredient | % w/v |
|---|---|
| Cyclosporine | 0.10 |
| Polyoxyl 15 Hydroxystearate | 0.50 |
| Carbomer Homopolymer Type B | 0.10 |
| Glycerin | 2.5 |
| Sodium Hydroxide | qs, pH 6.5-7.5 |
| Purified Water | qs. 100.00 |

Part 1 was prepared by melting SOLUTOL HS 15 polyoxyl 15 hydroxystearate and heating it to 60-70° C. Cyclosporine was added and mixed until completely dissolved.

Part 2 consisted of the remaining ingredients (carbomer and glycerin) and was prepared by adding glycerin to water followed by dispersing carbomer in the solution and pH adjustment with sodium hydroxide to pH 6.55.

After heating Part 2 to 70-75° C. a stator-rotor OMNI mixer was introduced in the vessel, and, while mixing, Part 1 was added rapidly and mixed at high shear for about 10 minutes. At the end of mixing the temperature of the product was 53° C. The OMNI mixer was removed and replaced by a magnetic stir bar. The product was mixed to room temperature before sampling for particle size analysis using a Horiba LA950 laser light scattering instrument. The mean particle size obtained was 313 nanometers.

This example confirmed that substances that are solid at room temperature can be used as solvents for cyclosporine when melted at higher temperatures.

### EXAMPLE 8

In this example, the stability of one of the disclosed formulations was assessed. Samples of the formulation described in Example 2 were stored continuously at 2-8 ° C., 25 ° C., or 40 ° C. and particle size was measured at various time points. The table below shows the particle size (mean particle size in nanometers) of the cyclosporine particles in the formulation determined at initial time (time 0), 4 weeks, 18 weeks, and 39 weeks.

### Mean Particle Size (Nanometers)

| | Temperature, ° C. | | |
|---|---|---|---|
| Time | 2-8 | 25 | 40 |
| Initial | 138 | 138 | 138 |
| 4 weeks | | | 235 |
| 18 weeks | 179 | 260 | 280 |
| 39 weeks | 264 | 740 | |

### EXAMPLE 9

In this example, the following samples (i.e., Samples 1-4) were tested by X-ray diffraction (XRD) to determine the crystallinity of the particles.

Sample 1: Cyclosporine API used in all batches.

Sample 2: sample of the formulation described in Example 6 above. The sample was tested after about 6 months storage at ambient room temperature.

Sample 3: sample of the following formulation, which was about one month old at ambient room temperature:

| Ingredient | % w/v |
|---|---|
| Cyclosporine | 0.10 |
| Tyloxapol | 0.025 |
| PEG300 | 2.0 |
| Glycerin | 2.0 |
| Benzalkonium Chloride | 0.010 |
| NaOH/HCl | adjust pH to 5-7 |
| Purified Water | q.s. 100.0 |

Sample 4: sample of the following formulation, which was about 8 months old at ambient room temperature:

| Ingredient | % w/v |
|---|---|
| Cyclosporine | 0.10 |
| Hypromellose 2910 | 0.50 |
| Polysorbate 80 | 0.050 |
| PEG300 | 2.0 |
| Glycerin | 2.0 |
| Benzalkonium Chloride | 0.010 |
| NaOH/HCl | adjust pH to 7-7.4 |
| Purified Water | q.s. 100.0 |

The results showed Sample 1 to match the known pattern of cyclosporine A Form 1. The solids filtered from Samples 2, 3, and 4 were amorphous. The dispersed particles appeared to maintain their amorphous nature after prolonged storage and not just immediately after preparation.

Although the invention has been described with reference to the above example, it will be understood that modifications and variations are encompassed within the spirit and scope of the invention. Accordingly, the invention is limited only by the following claims.

1.-30. (canceled)

31. A method of producing a suspension of cyclosporin comprising mixing a cyclosporin solution into an aqueous mixture to precipitate cyclosporin particles having particle size of 20 μm or less as determined by laser light scattering; wherein the cyclosporin solution comprises cyclosporin that is completely dissolved in polyethylene glycol prior to being mixed with the aqueous mixture; wherein the aqueous mixture comprises a dispersing agent, a suspending agent, and an aqueous vehicle; and wherein the suspension of cyclosporin is oil-free and fat free.

32. The method of claim 31, wherein the cyclosporin is cyclosporine.

33. The method of claim 31, wherein the cyclosporin is about 0.005 to about 1.0% w/v of the suspension of cyclosporin.

34. The method of claim 31, wherein the dispersing agent is a surfactant.

35. The method of claim 34, wherein the surfactant is polysorbate 80.

36. The method of claim 34, wherein the surfactant is polysorbate 60.

37. The method of claim 34, wherein the surfactant is polysorbate 40.

38. The method of claim 34, wherein the surfactant is polysorbate 20.

39. The method of claim 34, wherein the surfactant is polyoxyl 40 stearate.

40. The method of claim 34, wherein the surfactant is polyoxyl 15 hydroxystearate.

41. The method of claim 34, wherein the surfactant is a poloxamer.

42. The method of claim 34, wherein the surfactant is tyloxapol.

43. The method of claim 34, wherein the surfactant is POE 35 castor oil.

44. The method of claim 31, wherein the surfactant is a hydrophilic surfactant.

45. The method of claim 31, wherein the suspending agent is an acrylic acid homopolymer, an acrylic acid copolymer, an acrylic acid interpolymer, polycarbophil, a soluble cellulose derivative, polyvinyl alcohol, polyvinypyrrolidone, hyaluronic acid, chondroitin sulfate, gellan, a natural natural gum, a salt thereof, or a combination thereof.

46. The method of claim 31, wherein the suspension of cyclosporin comprises cyclosporin particles of 5 μm or less.

47. The method of claim 31, wherein the cyclosporin particles are amorphous.
